(19) United States
(12) Patent Application Publication
(10) Pub. No.: US 2020/0170825 A1
(43) Pub. Date: Jun. 4, 2020

(54) FOOTWEAR TO CORRECT PRONATION ISSUES

(71) Applicant: Footwear Unlimited Inc., Fenton, MO (US)

(72) Inventors: Louis Aquino, St. Louis, MO (US); Fabio Lucca, Valley Park, MO (US)

(21) Appl. No.: 16/701,455

(22) Filed: Dec. 3, 2019

Related U.S. Application Data
(60) Provisional application No. 62/774,656, filed on Dec. 3, 2018.

(51) Int. Cl. A61F 5/14; A43B 17/02

(52) U.S. Cl. CPC A61F 5/14; A43B 17/02

(57) ABSTRACT

A shoe comprising an inclination element that defines a gradient or inclination to correct pronation issues. The inclination element may be integrally formed with the shoe's insole, the midsole, the outsole, or any combination thereof. The shoe may further comprise a wedge member, which may be coupled to the midsole and define another gradient to properly position the heel of the foot during foot stride. The insole may also comprise a first cushioning material and a second rigid material. The gradient or gradients may be applied so as to roll the arches of the wearer's foot outward when walking and thereby correcting the normal inward or downward pull of such arches. This gradient may also be applied solely to the heel portion of the footwear so as to properly position the heel of a person's foot during foot stride thereby correcting and realigning posture.

### CROSS-REFERENCE TO RELATED APPLICATIONS

This application claims priority to U.S. Provisional Patent Application No. 62/774,656, filed Dec. 3, 2018, to Louis Aquino and Fabio Lucca, entitled “Footwear to Correct Pronation Issues,” currently pending. The entire disclosure, including the specification and drawings, of the above-referenced application is incorporated herein by reference.

### FIELD OF THE INVENTION

The present invention relates generally to footwear, and more particularly to an improved shoe for automatically correcting pronation issues.

### BACKGROUND OF THE INVENTION

Pronation is the natural movement of the foot that occurs during foot stride while running or walking. It refers to the foot's natural way of moving from side-to-side when a person is walking or running. It occurs as the weight of a person is transferred forward from the heel to the ball of the foot as a person goes through their walking or running stride. Pronation also occurs while standing and, in this situation, pronation refers to the amount that the foot rolls inward toward the arch.

A person's gait can show a pattern of either neutral pronation, overpronation, or underpronation. Underpronation is commonly referred to as “supination.” The stresses of overpronating or supinating have been linked to a greater risk of injury. Overpronation is when the arches of the foot roll inward or downward excessively and this is often referred to as flat feet. A person's foot normally rolls a little inward with each step. From time to time, as a person walks or runs, his or her heel strikes the ground, the person's arch begins to flatten and cushion the shock. Through the stride, the person's weight transfers forward from the heel to the ball of the foot while also shifting to the outside of the foot and then back to the big toe. If a person has a neutral gait, their foot should begin to roll outward with toe-off.

The normal biomechanics of the foot absorb and direct a person's weight throughout the gait. Overpronation puts a person at an increased risk of developing specific injuries. This is because it disrupts the body's natural alignment and causes increased impact when the foot strikes the ground.

There are many possible causes for overpronation, but researchers have not yet determined one underlying cause. Overpronation may occur for a wide variety of anatomical reasons. Regardless of the specific cause, overpronation may cause injuries such as shin splints, bunions, heel pain, plantar fasciitis, chronic lower back pain, stress fractures in the foot or lower leg, tendonitis, heel spurs and much more.

Overpronation can be treated conservatively with custom-made orthotic inserts. These orthotic inserts are custom designed with appropriate arch support and medial rear foot posting and focused primarily on impact relief relative to a person's foot to prevent overpronation. Custom orthotic inserts are slipped into shoes on top, and in addition to, of the insole already present in the shoe and further constrict the space of the shoe into which a person's foot will be inserted. Although custom orthotic inserts do provide relief, they can also cause other problems such as cramping of the foot into the now smaller space of the shoe. In addition, orthotic inserts are typically tailored and fitted for one specific pair of shoes and a person may need orthotic inserts for each pair of shoes that are being worn so that pronation can be controlled and comfort can be achieved for each pair of shoes. Custom orthotic inserts are also expensive, which can be compounded by the need to have orthotic inserts for each pair of a user's shoes.

It is therefore desirable to design a shoe from the beginning so as to have a gradient or inclination element already built into the shoe or portion of the shoe, such as the insole, midsole, and/or outsole, so as to correct overpronation and other pronation issues. Such shoe being a cost-effective alternative to the treatment of overpronation and supination issues, without having expensive custom-made orthotic inserts placed on top of an existing insole. Aftermarket insoles also take up more space within the shoe, thereby decreasing the space into which a person's foot could be positioned. Such shoe further providing a customized solution to pronation issues built into a particular shoe. The shoes can be selectively manufactured with varying degrees of gradient or inclination, depending on the degree of pronation to be prevented.

### SUMMARY OF THE INVENTION

Disclosed herein is a shoe for correcting pronation issues. The shoe can comprise a sole element and a first inclination element. The sole element may comprise an insole coupled with a midsole and an outsole coupled with the midsole. The first inclination element may be located adjacent to a heel portion of the shoe and coupled with the sole element. Further, the insole may comprise a heel portion, a midfoot area, a forefoot area, and an arch area, and the first inclination element may define a first gradient to correct pronation issues. The first gradient can slope from the inside part of the foot towards the outside part of the foot in the range from approximately 1° to approximately 6°.

The first inclination element may be integrally formed with the insole, the midsole, and/or the outsole. Further, the shoe may further comprise a wedge member that can define a second gradient and be coupled to the midsole. The second gradient can slope from the inside part of the foot towards the outside part of the foot in the range from approximately 1° to approximately 6°. In one embodiment, the wedge member can be integrally formed with the shoe.

In another embodiment, the shoe may further comprise a second inclination element located adjacent to a heel portion of the shoe and coupled with the sole element to correct pronation issues. The second inclination element may be integrally formed with the midsole and/or the outsole. In such embodiment, the first inclination element may define a gradient of approximately 2°, and the second inclination element may define a gradient of approximately 2°.

In yet another embodiment, the insole of the shoe may comprise a first material and a second material. The first material may comprise of a cushioning material, and at least a portion of the heel portion and/or the midfoot area and the forefoot area may be comprised of the first material. The second material may comprise of a rigid material, and the second material may circumscribe a perimeter defined by the heel portion and/or extend into at least a portion of the midfoot area and/or the arch area.

The shoe may further comprise a two-piece outsole coupled with a bridge support. Further, the show may comprise a multi-piece midsole, wherein the insole mates with the midsole. The shoe may be a high heel shoe and/or a sandal.

### BRIEF DESCRIPTION OF DRAWINGS

For a better understanding of the present invention, reference may be made to the following accompanying drawings.

FIG. 1 is a perspective view showing a foot experiencing overpronation.

FIG. 2 is a perspective view showing a foot experiencing natural pronation using a wedge member for correcting overpronation constructed in accordance with the teachings of the present invention.

FIG. 3 is a side elevational view of one embodiment of the present invention.

FIG. 4 is an exploded side elevational view of the embodiment of FIG. 3 showing the use of a wedge member for correcting overpronation.

FIG. 5 is a top view of an insole constructed in accordance with the teachings of the present invention.

FIG. 6 is a bottom view of the insole of FIG. 5.

FIG. 7 is a side view of the insole of FIGS. 5 and 6.

FIG. 8 is a partial rear perspective view of the insole of FIGS. 5-7.

FIG. 9 is a side view of one embodiment of the present invention utilizing the insole of FIGS. 5-8 in a particular shoe construction.

FIG. 10 is an exploded side elevational view of the embodiment of FIG. 9 showing the use of the insole of FIGS. 5-8.

FIG. 11 is a partial rear perspective view of a shoe showing the present invention built directly into the heel portion of a shoe.

FIG. 12 is a partial rear perspective view similar to FIG. 11 showing the present invention built directly into the heel portion of a sandal.

FIG. 13 is a partial rear perspective view of another embodiment of the present invention showing the inclination element built into both the insole and the midsole.

FIG. 14 is a partial side perspective view similar to FIG. 13 showing other variations of the present inclination element built into a particular shoe.

While the present disclosure is susceptible to various modifications in alternative forms, a specific embodiment of the present invention is shown by way of example in the present drawings and will herein be described in more detail. It should be understood however, that the present drawings and detailed description presented herein are not intended to limit the disclosure to the particular embodiment disclosed, but to the contrary, the intention is to cover all modifications, equivalents and alternatives falling within the spirit and scope of the present disclosure.

### DETAILED DESCRIPTION OF THE INVENTION

The invention will now be described with reference to the drawing figures, in which like reference numerals refer to like parts throughout. For purposes of clarity in illustrating the characteristics of the present invention, proportional relationships of the elements have not necessarily been maintained in the drawing figures.

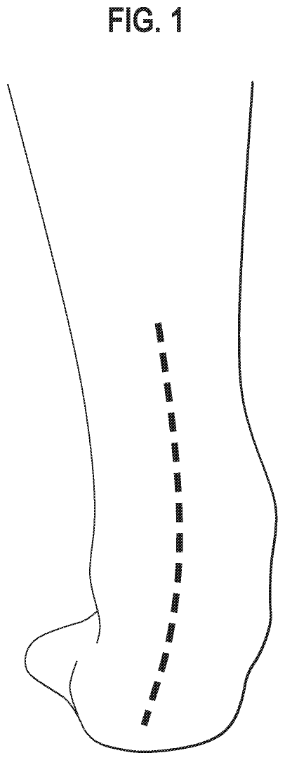

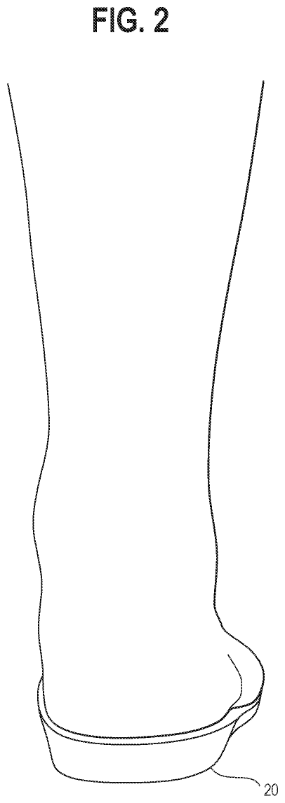

The present invention is specifically directed to constructing a shoe that provides the required inclination or gradient to properly position the heel bone within the heel portion of the shoe so as to correct for overpronation or other pronation issues. Heel position can be important for solving pronation issues, because as a person goes through a stride, his or her heel generally contacts the ground first and weight is transferred from the heel to the ball of the foot. FIG. 1 depicts a foot experiencing overpronation. Correcting for overpronation or other pronation issues can be accomplished in many different ways. For example, FIG. 2 illustrates a wedge member 20 utilized to define or provide the proper gradient or inclination to correct overpronation, such that the foot experiences natural pronation.

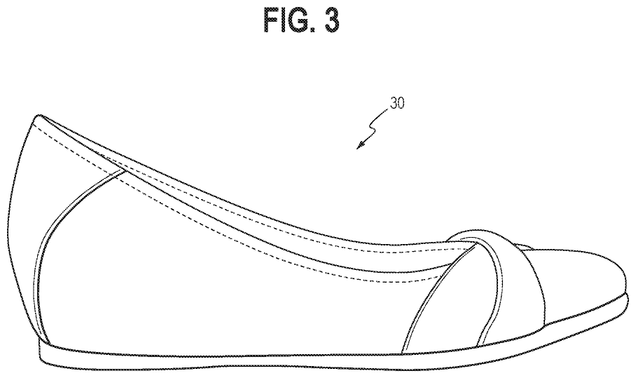

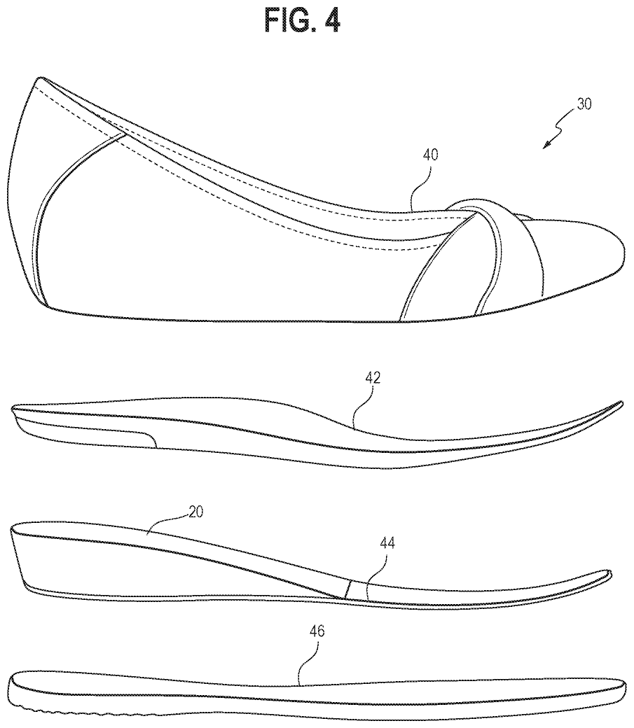

FIG. 3 illustrates a shoe 30 constructed in accordance with the teachings of the present invention. As best illustrated in FIG. 4, the shoe 30 can comprise the wedge member 20, an upper 40, and a sole element generally comprising an insole 42, a midsole 44 and an outsole 46. In one embodiment, the insole 42 may be attached or coupled to the midsole 44. In another embodiment, the midsole 44 may be attached or coupled to the outsole 46. In yet another embodiment, the wedge member 20 can be coupled to the midsole 44. However, it will be understood that the wedge member can be coupled to the insole 42, midsole 44, and/or the outsole 46. The wedge member 20, which can be located adjacent to the heel portion of the shoe 30, is specifically constructed so as to provide a gradient or inclination which slopes towards the outside part of the foot or away from the ankle. This gradient or inclination can be anywhere in the range from approximately 1° to approximately 6°, in one embodiment, depending upon the amount of overpronation necessary to correct. The wedge member 20 is specifically built into or integral formed with the shoe 30 so that a person wearing the shoe 30 will have proper support during foot strike so as to overcome the overpronation issue and to improve the posture of the individual wearing the shoe 30.

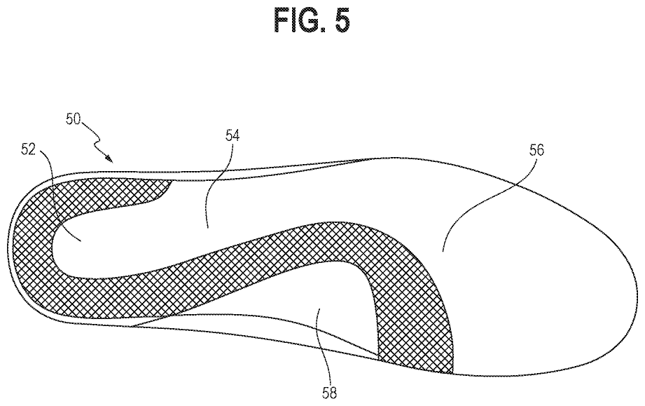

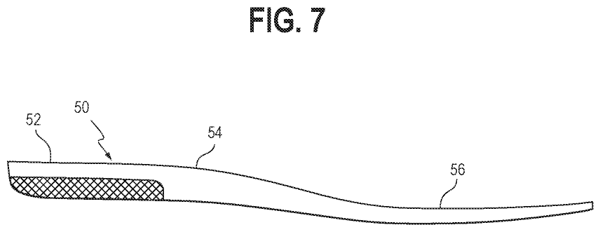

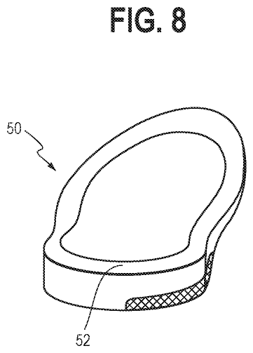

FIGS. 5-8 depict another aspect of the present invention wherein the inclination element, which may be located adjacent the heel portion of a shoe (not shown), may be utilized to correct for overpronation by defining an inclination or gradient. In one embodiment, the location of the inclination element adjacent to the heel portion of the shoe aids in proper heel positioning. In one embodiment, the inclination element may be coupled with the sole element of a shoe. In another embodiment, the inclination element may be built into or integrally formed with an insole 50 of any particular shoe. However, it will be understood that the inclination element may be integrally formed with any portion of the sole element, including a midsole (not shown) or an outsole (not shown). As best illustrated in FIG. 5, the insole 50 may generally comprise a heel portion 52, a midfoot area 54, forefoot area 56, and an arch area 58. FIGS. 5-8 best illustrate, with respect to another embodiment, the gradient or inclination provided by present invention which slopes towards the outside part of the foot or away from the ankle, as will be hereinafter further explained. Here again, the inclination element is built into the heel portion 52 of the insole 50 as best illustrated in FIG. 8. The inclination again slopes from the inside part of the foot towards the outside part of the foot so that the heel bone of a person wearing the insole 50 is properly positioned in a more upright position. Here again, this inclination or gradient can vary from approximately 1° to approximately 6°, in one embodiment, or more depending upon the amount of overpronation necessary to correct the situation.

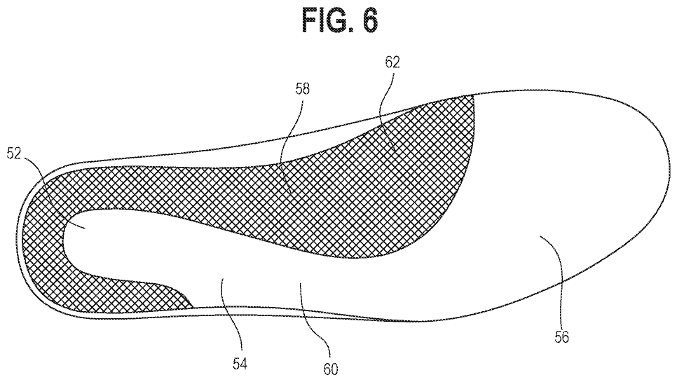

In still another aspect of the present invention, the insole 50 can be constructed from two different types of materials as best illustrated in FIG. 6. More particularly, the insole 50 can include a soft cushioning material 60 which includes portions of the heel portion 52 and midfoot area 54 and all of the forefoot area 56. Whereas a second rigid material 62 is harder and used to circumscribe the perimeter of the heel portion 52 and extend into the midfoot area 54 and arch area 58, as illustrated. This harder material 62 provides additional strength and stability to the insole 50 and again contributes to the inclination element built into the insole 50 so as to properly position the heel bone of a person wearing the present insole. The insole 50 can be utilized in any shoe construction and again provides an alternative to buying a specific, expensive custom-made orthotics.

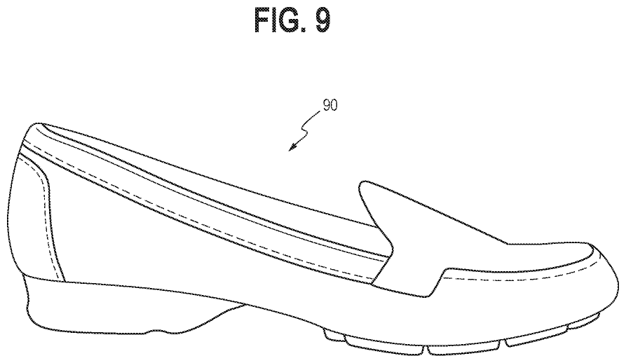

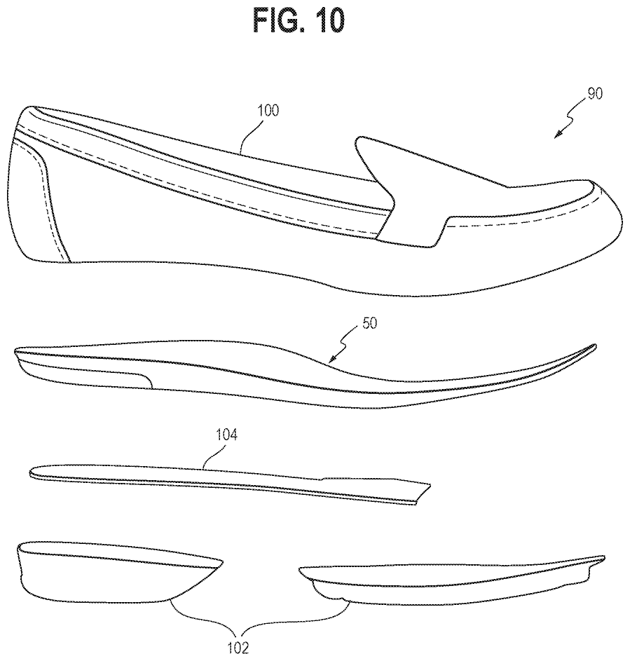

For example, as illustrated in FIGS. 9 and 10, the insole 50 can be utilized in a particular shoe 90 which includes an upper 100, the insole 50, a two-piece outsole 102 and a bridge support member 104. It is also recognized and anticipated that the insole 50 can be used in a shoe having a single outsole construction and a single or multi-piece midsole construction so long as the insole is positionable on and mates with the midsole.

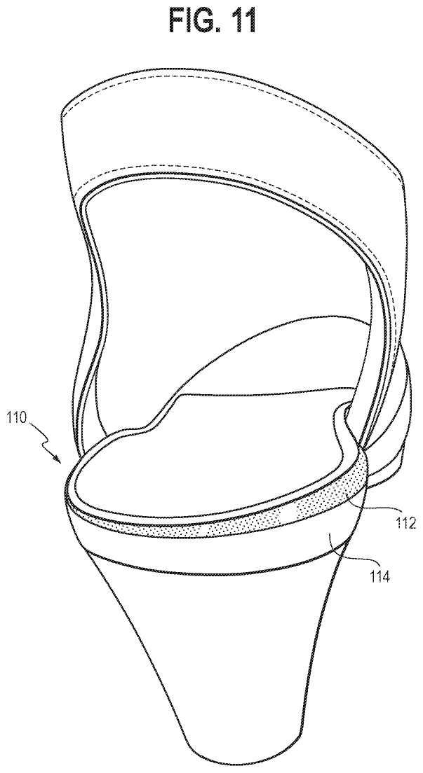

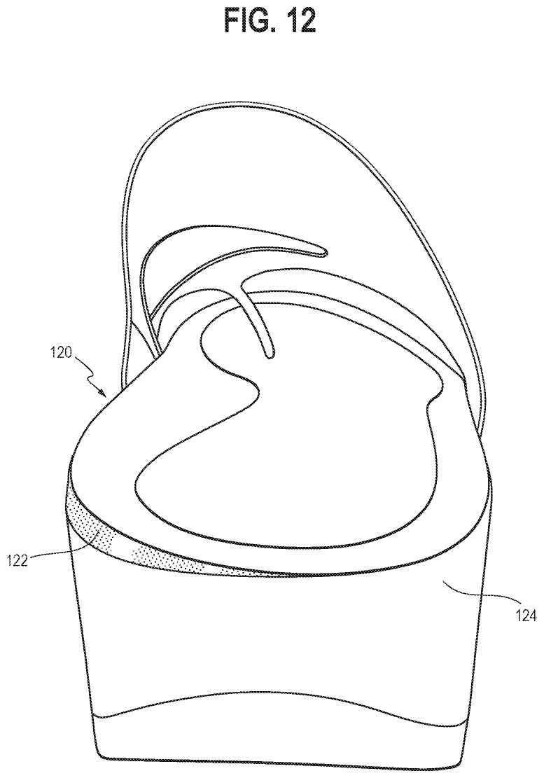

Still further, in another aspect of the present invention, FIG. 11 illustrates a women's high heel shoe 110 wherein the inclination element 112 is built directly into the upper heel portion 114 of the shoe 110. Here again, the inclination element 112 can define a gradient or inclination in the range anywhere from approximately 1° to approximately 6°, in one embodiment, sloping towards the outward part of the shoe 110. The same is likewise true with respect to the sandal type shoe 120 illustrated in FIG. 12 wherein the inclination element 122 is again built directly into the heel portion 124 of the shoe so as to slope towards the outside part of the foot.

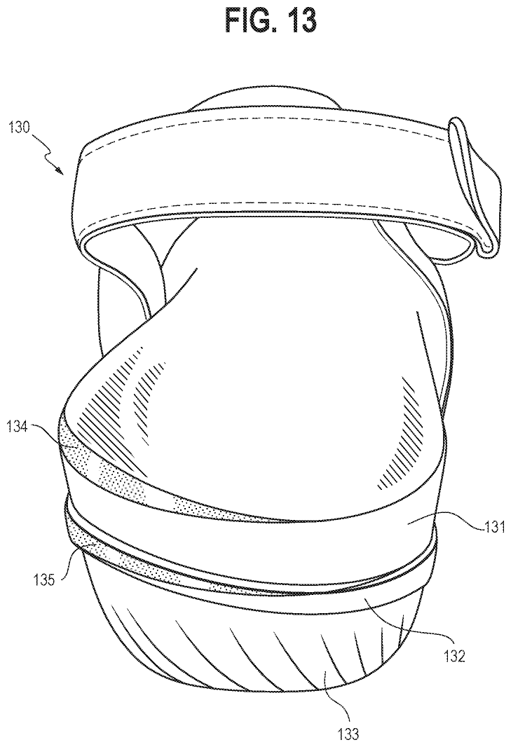

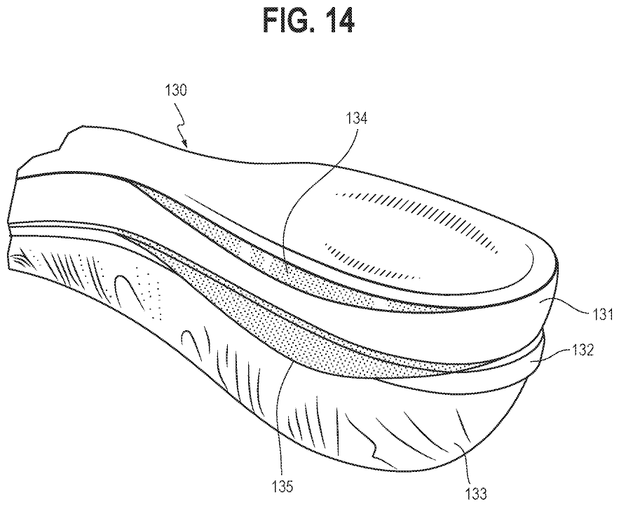

It will be recognized and anticipated that the inclination element built into a particular shoe can be accomplished by inclining both the insole and midsole or the midsole and outsole, or the insole and outsole, or the insole, midsole and outsole, in any combination to achieve the necessary inclination. These constructions aid in proper heeling positing and provide extra support and stability that minimize the impact of each foot stride. For example, FIG. 13 illustrates a particular shoe 130 generally comprising an insole 131, a midsole 132, and an outsole 133. FIG. 14 depicts another view of shoe 130. As best illustrated in FIG. 13, a first inclination element 134 is associated with the insole 131, and a second inclination element 135 is associated with the midsole 132. However, it will be understood that yet another inclination element (not shown) can be associated with the outsole 133. The second inclination element 135 may be located adjacent the heel portion 136 of the shoe 130. According to one embodiment, the insole 131 may be inclined a first number of degrees and the midsole 132 may be inclined a second number of degrees such that the combination of the first inclination element 134 and the second inclination element 135 provides the total inclination desired. In this case, for example, the first inclination element 134 is associated with the insole 131 may define approximately a 2° gradient, and the second inclination element 135 is associated with the midsole 132 the midsole may define approximately a 2° gradient thereby providing a total of approximately a 4° gradient for the shoe 130. The same combination is likewise true with respect to inclining the midsole 132 and outsole 133 and/or inclining the insole 131 and outsole 133. Any combination is possible including inclining the insole 131, midsole 132, and outsole 133 of the shoe 130 to achieve the overall desired gradient or inclination. Again, importantly, this inclination takes place only in the heel portion of the shoe for proper heel positioning, as illustrated in FIGS. 3-12. This inclination element built directly into a particular shoe provides posture from the heel to other parts of the body and improves the overall posture of the hips, knees and back of a particular individual.

Although the present invention has been described particularly for correcting an overpronation problem, the same theory, inclination and gradient can be used in various parts of the shoe as described above with respect to a supination problem. In this case, the inclination or gradient will run in the opposite direction, that is, towards the inward or inside part of the foot.

As a result, the present invention provides a cost-effective alternative to the treatment of overpronation and supination issues without having expensive custom-made orthotic inserts which are placed on top of the insole already constructed in a particular shoe and likewise takes up more space within the shoe thereby decreasing the space into which a person's foot will be positioned. In addition, the present invention provides a customized solution to pronation issues which is already built into a particular shoe and the shoes can be made with varying degrees of gradient or inclination.

Thus, there has been shown and described a shoe construction which corrects for various pronation issues without having to have customized, prescription ordered, orthotic inserts specifically constructed to solve a particular problem. As is evident from the foregoing description, certain aspects of the present invention are not limited by the particular details of the examples illustrated herein, and it is therefore contemplated that other modifications and applications, or equivalents thereof, will occur to those skilled in the art. The terms “having,” “including,” and similar terms as used in the foregoing specification are used in the sense of “optional” or “may include” and not as “required.” Many changes, modifications, variations, and other uses and applications of the present invention will, however, become apparent to those skilled in the art after considering the specification and the accompanying drawings. All such changes, modifications, variations, and other uses and applications which do not depart from the spirit and scope of the invention are deemed to be covered by the invention, which is limited only by the claims which follow.

What is claimed is:

1. A shoe comprising:
a sole element comprising an insole coupled with a midsole and an outsole coupled with the midsole; and
a first inclination element located adjacent to a heel portion of the shoe and coupled with the sole element;
wherein:
the insole comprises a heel portion, a midfoot area, a forefoot area, and an arch area; and
the first inclination element defines a first gradient to correct pronation issues.

2. The shoe of claim 1, wherein the first gradient slopes from the inside part of the foot towards the outside part of the foot in the range from approximately 1° to approximately 6°.

3. The shoe of claim 1, wherein the first inclination element is integrally formed with the insole.

4. The shoe of claim 3 further comprising:
a wedge member coupled to the midsole;
wherein the wedge member defines a second gradient.

5. The shoe of claim 4, wherein the second gradient slopes from the inside part of the foot towards the outside part of the foot in the range from approximately 1° to approximately 6°.

6. The shoe of claim 4, wherein the wedge member is integrally formed with the shoe.

7. The shoe of claim 1, wherein the first inclination element is integrally formed with the midsole.

8. The shoe of claim 1, wherein the first inclination element is integrally formed with the outsole.

9. The shoe of claim 1 further comprising a second inclination element located adjacent to a heel portion of the shoe and coupled with the sole element to correct pronation issues.

10. The shoe of claim 9, wherein the second inclination element is integrally formed with the midsole.

11. The shoe of claim 9, wherein the second inclination element is integrally formed with the outsole.

12. The shoe of claim 9, wherein:
the first inclination element defines a gradient of approximately 2°; and
the second inclination element defines a gradient of approximately 2°.

13. The shoe of claim 1, wherein:
the insole comprises a first material and a second material;
wherein:
the first material is comprised of a cushioning material; and
the second material is comprised of rigid material.

14. The shoe of claim 13, wherein:
at least a portion of the heel portion comprises the first material;
at least a portion of the midfoot area comprises the first material; and
the forefoot area comprises the first material.

15. The shoe of claim 13, wherein:
the second material circumscribes a perimeter defined by the heel portion;
the second material extends into at least a portion of the midfoot area; and
the second material extends into at least a portion of the arch area.

16. The shoe of claim 1 further comprising a two-piece outsole coupled with a bridge support.

17. The shoe of claim 1 further comprising:
a multi-piece midsole;
wherein the insole mates with the midsole.

18. The shoe of claim 1, wherein the shoe is a high heel shoe.

19. The shoe of claim 1, wherein the shoe is a sandal.
